SEQUENCE LISTING (2 sequences)

SEQ ID NO: 1 - DNA, 366 bp, Artificial Sequence
  Synthetic
atggactactacagaaagcacgccgccgtgatcctggctaccctgtccgtgttcctgcacatcctgcatagcttccccgacggcgagttcacaacccaggactgccctgagtgcaagctgagagagaacaagtacttcttcaagctgggcgtgcccatctaccagtgcaagggctgctgcttctcccgggcctatcctacccctgcccggtccagaaagaccatgctggtgcccaagaacatcacctccgagtctacctgctgcgtggccaaggccttcatcagagtgaccgtgatgggcaacatcaagctggaaaaccacacccagtgctactgctccacctgttaccaccacaagatctgatga

SEQ ID NO: 2 - DNA, 513 bp, Artificial Sequence
  Synthetic
atggaaacactgcagggcctgctgctgtggatgctgctgtctgtgggcggcgtgtgggcttctagaggacctctgaggcccctgtgccggcctatcaatgctaccctggccgccgagaaagaggcctgccctatctgcatcaccttcaccacctccatctgcgccggctactgcccctccatggtgcgagtgatgccagccgctctgcctgccattcctcagcccgtgtgcacctacagagagctgagattcgcctccatccggctgcctggatgtcctcctggcgtggaccctatggtgtccttccctgtggccctgtcttgccactgcggcccctgtcagatcaagaccaccgactgcggcgtgttccgggatcagcctctggcatgtgcacctcaggcctccagctcctccaaggaccctccatctcagcccctgacctccacctctacccctacacctggcgcctctcggagatcctctcaccccctgcctatcaagacctcctgatga

(19) United States
(12) Patent Application Publication
(10) Pub. No.: US 2023/0242608 A1
(43) Pub. Date: Aug. 3, 2023

(54) METHOD FOR OBTAINING A MAMMALIAN CELL LINE THAT EXPRESSES A RECOMBINANT EQUINE CHORIONIC GONADOTROPIN (RECG), THE RECOMBINANT CELL LINES PRODUCING RECG, LARGE-SCALE RECG PRODUCTION METHOD, RECG, FORMULATIONS CONTAINING RECG, NUCLEIC ACIDS ENCODING FOR RECG AND USES

(71) Applicants: Natalia CEAGLIO, Santo Tome, Santa Fe (AR); UNIVERSIDAD NACIONAL DEL LITORAL, Santa Fe, Santa Fe (AR); CONSEJO NACIONAL DE INVESTIGACIONES CIENTÍFICAS Y TÉCNICAS (CONICET), Caba (AR); BIOTECNOFE S.A., Santa Fe, Santa Fe (AR)

(72) Inventors: Natalia Ceaglio, Santo Tome, Santa Fe (AR); Claudio Prieto, Santa Fe, Santa Fe (AR); Diego Fontana, Santa Fe, Santa Fe (AR); Celeste Rodriguez, General Ramirez, Entre Rios (AR); Belén Tardivo, Santa Fe, Santa Fe (AR); Sebastián Antuna, Franck, Santa Fe (AR)

(21) Appl. No.: 17/790,385

(22) PCT Filed: Dec. 23, 2020
(86) PCT No.: PCT/EP2020/087779

§ 371 Date: Jun. 30, 2022

(30) Foreign Application Priority Data
Dec. 30, 2019 (AR) P 20190103911

(51) Int. Cl. C07K 14/59; C12N 15/86; A61P 15/08; A61K 9/19

(52) U.S. Cl. CPC C07K 14/59; C12N 15/86; A61P 15/08; A61K 9/19; C12N 2740/16043; A61K 38/00

(57) ABSTRACT

The present invention describes a method for obtaining a mammalian cell line that expresses a recombinant equine chorionic gonadotropin (reCG) hormone. A cell line expressing reCG, a large-scale method for producing reCG, a reCG with a higher biological activity regarding PMSG, formulations containing reCG, nucleic acids encoding reCG and uses are also described.

### FIELD OF THE INVENTION

The present invention belongs to the field of biotechnology, specifically to the development of protein hormones through recombinant DNA technology and their production in mammalian cells.

### BACKGROUND OF THE INVENTION

Equine chorionic gonadotropin (eCG) is a member of the glycoprotein hormone family together with luteinizing hormone (LH), follicle-stimulating hormone (FSH) and thyroid stimulating hormone (TSH) (Murphy and Martinuk, 1991). As eCG is produced by trophoblast cells of the endometrial cups of pregnant mares, it was originally called Pregnant Mare Serum Gonadotropin (PMSG). The eCG plays an important role in the maintenance of early gestation (during first three months), indirectly stimulating progesterone production by corpus luteum until the placenta can secrete it on its own. The concentration of eCG secreted by trophoblasts reaches its maximum concentration around the day 50 of pregnancy and then starts to decline progressively (Allen and Moor, 1972).

The eCG has two distinctive characteristics compared to the other glycoprotein hormones. On the one hand, in species other than equine, eCG shows high FSH- and LH-like activity and has a high affinity for the receptors of these hormones (Combarnous et al., 1984). On the other hand, it exhibits a high carbohydrate content, which constitutes 45% of its total molecular weight. This last property determines the long circulating half-life of eCG, of about six days. Due to both characteristics, eCG is used in veterinary medicine to control reproductive activity of different types of livestock, including cattle, sheep, goats and pigs (Rensis and L6pez-Gatius, 2014).

Like other members of the glycoprotein hormone family, eCG is a heterodimeric protein composed by two different non-covalently linked subunits, named a and 13. The a subunit is common to all members of the family and it is encoded by a single gene, while different genes encode the 13 subunits, which confer specificity to heterodimers (Stewart and Allen, 1976).

The a subunit is composed by 96 amino acids and has two N-glycosilation sites located at Asn56 and Asn82, while the 13 subunit is composed by 149 amino acids and has an only N-glicosilation site at Asn13. Furthermore, 13 subunit has a carboxyl terminal peptide (CTP) composed by 28 amino acids (122-149) that contains 12 O-glycosylation sites in Sero Thr residues (Bousfield and Butnev, 2001). Both subunits contain multiple intramolecular disulfide bonds and their assembly occurs mainly in the endoplasmic reticulum, which represents the limiting step in the dimer secretion process (Hoshina and Beirne, 1982). In equines, both placental CG and pituitary LH 13 subunits are encoded by the same gene (Sherman et al., 1992). However, the eCG is composed by higher and more branched carbohydrate content than eLH. Both hormones differ considerably in their N-glycan terminations. The eCG has glycans capped with N-acetylneuraminic acid (sialic acid), while eLH exhibited sulfated N-acetylgalactosamine (SO4-⁴-GalNAc) glycans. The remarkable difference in their molecular weight is essentially due to the presence of longer di-sialylated poly-N-acetylamines O-glycan structures in the eCG (Smith et al., 1993). The high sialic acid content of eCG is responsible for its exceptional circulating half-life, since this residue reduces both glomerular filtration and liver metabolism.

Currently, the products available in the market are partially purified eCG preparations from blood of pregnant mares (PMSG), comprising many disadvantages. On the one hand, they show batch to batch variations, since the glycosylation profile varies between animals and between sera at different stages of gestation. On the other hand, PMSG may also contain contaminants with potential health risks. This goes against the current trend of regulatory entities to obtain safer veterinary products, free of viruses, prions and other contaminating proteins. Last but not least, the practice by which the serum containing eCG is obtained from the pregnant mare involves the weekly extraction of 10 liters of blood and the subsequent induction of manual abortion by reaching the uterus and bursting the amniotic sac. The practice to which the animal is subjected compromises animal welfare, and it is completely questionable from the bioethical point of view: it is a bloody process which may generate significant anemias and, in some cases, ends with the animal's life.

For this reason, the development of a recombinant eCG (reCG) as a substitute of PMSG is reasonable. Several efforts to produce recombinant eCG have been reported in different hosts. On the one hand, Legardinier et al. (2005) described the production of eCG in two insect cell lines, Sf9 and Mimic™, the latter being a cell line derived from the former that has been modified to express different genes from mammalian glycosyltransferases.

Nevertheless, the produced hormone did not demonstrate FSH/LH-like activity in in vivo rat models, which the authors attributed to its extremely short circulating half-life caused by the absence of terminal sialic acid in the oligosaccharide chains. On the other hand, Ubach et al. (2009) and Ingles et al. (2012) described the expression and purification of eCG in Pichia pastoris; nevertheless, the recombinant hormone did not present bioactivity in the in vivo bioassay in female rats. Again, these results were correlated with the short circulating half-life of the recombinant hormone, since only 1% of the injected protein could be detected in serum after 90 min. This rapid clearance could be explained by the activation of the mannan-binding lectin pathway that occurs after the injection of a protein with high mannose content. These results reveal the importance of a correct glycosylation profile for eCG to exhibit in vivo bioactivity and, therefore, the importance of the correct host selection for its recombinant expression.

The development of recombinant versions of eCG in CHO cells, including the CHO DG44 cell line, has been reported in publications and patents. WO2017112987A 1 patent application describes the use of this CHO cell line and the obtaining of a reCG with the expected glycosylation profile according to the host used. Despite this, commercial versions of reCG are not yet available on the market. This result evidences a problem to be solved, which consists in the fact that large amounts of recombinant eCG (or recombinant gonadotropins in general) cannot be obtained in an “efficient way”, or with a glycosylation profile like that of natural eCG, which guarantees its in vivo biological activity (Hesser, 2011). In this way, the challenge is to develop a production system that exhibits high productivity to obtain sufficient quantities of reCG satisfying the high demand (since the hormone is used in different types of livestock) and, therefore, results in lower production costs, which is the limiting factor for recombinant hormone to prosper in the market. WO2017112987A1_2017 describes the use of the DHFR-MTX gene amplification system, obtaining a production of 18 IU/ml (in serum-free medium). This value fails to achieve a profitable process with respect to the current PMSG production process.

### BRIEF DESCRIPTION OF THE DRAWINGS

FIG. 1 is a schematic drawing of the assembly of third-generation lentiviral particles to obtain reCG-producing recombinant CHO-K1 cell lines.

FIG. 2 illustrates an SOS-PAGE followed by western blot where the concentration of reCG in supernatants from reCG-producing lines is evaluated using specific anti-reCG antibodies.

FIG. 3 illustrates an SOS-PAGE followed by western blot where the “apparent” productivity of preselected reCG-producing clones is evaluated.

FIG. 4 is a graph comparing the evolution of viable cell concentration, viability, lactate and glucose concentration along time of a culture of P5C3 clone in a 1 L bioreactor. Temperature and perfusion rate per day are shown. (t) represents bleeding of the bioreactor.

FIG. 5 is a structural characterization and purity analysis of (A) the PMSG molecule post RP-HPLC, (B) reCG RP-HPLC and (C) reCG HIC by SOS-PAGE, RP-HPLC and SEC-HPLC.

FIG. 6 shows an isoform profile analysis. (A) IEF followed by staining by colloidal coomassie blue. The isoform profile of the PMSG variant (commercial preparation A, Foli-G), and the CaptoB reCG eluate from a cell line and clone, respectively, were compared. (B) IEF followed by western blot of PMSG (commercial preparation A, Foli-G), and the CaptoB reCG eluate from a cell line and clone, respectively. (C) IEF followed by staining by colloidal coomassie blue. The isoform profile of PMSG (commercial preparation B, Novormon), reCG RP-HPLC and reCG HIC variants, respectively, was compared.

FIG. 7 shows the fluorescence emission spectrum of PMSG, reCG RP-HPLC and reCG HIC in sodium phosphate buffer solution.

FIG. 8 shows notched boxplot diagrams where the significant difference in Neu5Ac content (mol/mol protein) is shown.

FIG. 9 2-AB labelled N-glycan analysis of PMSG, reCG RP-HPLC and reCG HIC by WAX-HPLC. N-glycans were separated according to their charge.

FIG. 10. Comparative pregnancy rate.

### BRIEF DESCRIPTION OF THE INVENTION

The present invention describes a method for obtaining a mammalian cell line that expresses a recombinant equine chorionic gonadotropin (reCG) comprising the following steps:
a. providing coding sequences for reCG a and 13 subunits, optimized for their expression in mammalian cells;
b. introducing said coding sequences in lentiviral expression vectors;
c. producing lentiviruses containing reCG coding sequence;
d. transducing mammalian cells with said lentiviruses;
e. selecting the most suitable mammalian cell clone to produce reCG.

Said recombinant cell line exhibits a reCG production of at least 100 IU/ml in serum-free medium.

In a preferred embodiment of the invention, step a. of the method uses subunit sequences substantially similar to those a and 13 sequences of SEQ ID No 1 and SEQ ID No 2, respectively. In step b., lentiviral vectors consist of pl V vector containing EF-1a promoter. Step c. involves transient transfections of HEK293 cells with pREV, pVSVG, pMDL, pLV-reCG a and pLV-reCG 13 plasmids using cationic lipids as vehicles. Step d. involves transducing CHO-K1 cells.

In an alternative way, the method of the present invention involves performing two successive transduction events.

Another object of the present invention is to provide a mammalian cell line which is obtained by the preceding method and contains a nucleic acid encoding a recombinant equine chorionic gonadotropin (reCG) hormone, where the coding sequences for reCG a and 13 subunits comprise sequences substantially similar to SEQ ID No 1 y SEQ ID No 2. Preferably, said mammalian cell line is CHO-K1 and shows a reCG production of at least 100 IU/ml.

Another object of the present invention is to provide a method for producing a recombinant equine chorionic gonadotropin (reCG) hormone which comprises the following steps:
a. cultivating of said mammalian cell line in bioreactor in fetal calf serum-free medium for large-scale production of said reCG
b. harvesting the supernatant, and
c. purification.

This production method yields a reCG productivity of at least 100 IU/ml in serum-free medium.

In a preferred embodiment of the invention, said step a. involves cultivation in serum-free medium containing 50% commercial Excel302 medium and 50% phosphate-buffered saline. In said step c., the purification comprises a dye pseudo-affinity chromatography. Preferably, said dye pseudo-affinity chromatography uses CaptoBlue-Sepharose matrix. Alternatively, said purification step c. consists of tangential flow filtration and subsequent reCG concentration.

Optionally, said purification method also comprehends a HPLC purification step using a C4 column.

The reCG obtained by said production method comprises a specific activity (as in vivo potency units in relation to the protein mass determined by ELISA) of at least 6000 IU/mg.

Another object of the present invention comprises a nucleic acid encoding the reCG alpha subunit obtainable by the described production method, comprising a sequence substantially similar to SEQ ID No. 1. It also comprises a nucleic acid encoding the beta subunit of reCG of sequence substantially similar to SEQ ID No.2.

Another object of the present invention is a reCG hormone obtainable by the described production method that comprises a glycosylation profile with at least 3% neutral structures, and at least 3% tetra-sialylated structures. Preferably, said reCG comprises a glycosylation profile with at least 3% neutral structures, between 26 and 30% mono-sialylated structures, between 50 and 55% bi-sialylated structures, between 8 and 15% tri-sialylated structures and at least 3% tetra-sialylated structures.

Another object of the present invention is a pharmaceutical formulation comprising a therapeutically effective amount of the reCG described herein. In a preferred form, said formulation is liquid and is kept chilled, preferably at 5° C., without the need to be frozen for commercialization. In an alternative form, said formulation is lyophilized. In a preferred form, said formulation further comprises a sugar, a preservative, an antioxidant, mannitol, and an anti-aggregation agent. Preferably, said formulation comprises trisodium citrate dihydrate, citric acid monohydrate, arginine, sucrose, mannitol, L-methionine, poloxamer 188, m-cresol and water.

Another object of the present invention is a method for inducing ovulation in animals that comprises the administration of at least 140 IU/animal of reCG. Where the administration of said dose induces ovulation 48 h after its application.

### DETAILED DESCRIPTION OF THE INVENTION

The present invention describes a method for obtaining a mammalian cell line (clone) that expresses a recombinant equine chorionic gonadotropin (reCG) involving the following steps:
a. provide coding sequences for reCG a and 13 subunits optimized for their expression in mammalian cells
b. introduce said coding sequences in lentiviral expression vectors
c. produce lentiviruses containing reCG coding sequence
d. transduce mammalian cells with said lentiviruses
e. select the most suitable mammalian cell clone to produce reCG

Cell clones exhibiting a production of at least 100 IU/ml in serum-free medium in bioreactors at large-scale are obtained from the method described in the present invention.

One of the main characteristics of the method for obtaining a reCG-producing mammalian cell line is that it makes use of an innovative and optimized DNA sequence that encodes reCG. The sequence has been modified and optimized to be expressed in mammalian CHO cells. Preferably CHO-K1. The reCG comprises at least one alpha subunit and one beta subunit, and therefore the coding DNA sequences for both subunits have been optimized. Said coding sequences for each subunit are substantially similar or identical to the sequences SEQ ID No. 1 (a) and SEQ ID No. 2 (13).

These coding sequences for each subunit comprise small modifications in their nucleotides that make them optimal for the transcriptional, translational and post-translational machinery of CHO-K1 cells. After optimization, it was observed that the recombinant sequence of the eCG beta subunit showed a 82.2% homology with the non-optimized beta subunit sequence (“natural” eCG beta), whereas, regarding other recombinant sequences published in different patents, homology was lower (78%). It was observed that those nucleotide positions of our optimized recombinant sequence that differed both with the residues of the native sequence and with those of the optimized sequences published in other patents corresponded to 4% of the total. This percentage was enough to behave as a determining factor in obtaining higher levels of expression of the reCG with respect to those reported in the state of the art.

The method of obtaining a reCG-producing mammalian cell line of the present invention achieves stable reCG-producing cell lines by using third-generation lentiviral vectors as a genetic material transfer tool. The lentiviral vector used is pl V containing the EF-1a promoter. Vectors carrying each of the coding sequences for each subunit are called pLV-reCGa and pLV-reCGl3. In addition, said pLV contains a coding region for a puromycin resistance gene as a selection marker.

To produce the lentiviral particles, the present invention describes transient transfections of HEK293 cells with plasmids pREV, pVSVG, pMDL, pLV-reCGa and pLV-reCGl3, using cationic lipids as vehicles. Once the lentiviral particles are obtained, they are used to transduce mammalian cells (step d). Preferably said mammalian cells are CHO-K1 cells. Preferably, the process comprises two successive transductions.

Another object of the present invention comprises a mammalian cell line that has as part of its genome a nucleic acid encoding a recombinant equine chorionic gonadotropin hormone (reCG) where the coding sequences for the alpha and beta subunits of said reCG comprise substantially similar sequences to SEQ ID No. 1 and SEQ ID No. 2. These cells are developed by the method of obtaining a reCG-producing mammalian cell line of the present invention. Preferably, the cell line is CHO-K1. This cell line (clone) produces at least 100 IU reCG/ml in serum-free medium.

Another object of the present invention is to provide a method for producing a recombinant equine chorionic gonadotropin (reCG) hormone characterized because it comprises the following steps:
d. Cultivation of said mammalian cell line (clone) in bioreactor in serum-free medium for large-scale production of said reCG
e. supernatant harvest and
f. purification.

The method of production of a recombinant equine chorionic gonadotropin (reCG) of the present invention achieves a production of at least 100 IU/ml in serum-free medium. These high levels of production are due to several factors. First, to the high reCG productivity of the cell line transformed with the sequences SEQ ID No 1 and SEQ ID No 2 of the present invention, that has been additionally adapted and optimized for cultivation in bovine serum-free medium. The medium used for production is MC02 medium, which comprises 50% commercial Excel302 medium and 50% of a combination of salts, amino acids, 25 carbohydrates, etc., as described in Table 2.

Another important factor is the purification step. The state of the art describes complex and expensive purification processes that make the final product more expensive. The method of producing a recombinant equine chorionic gonadotropin hormone (reCG) of the present invention incorporates a purification step comprising dye-pseudoaffinity chromatography. In a preferred embodiment, the matrix used to carry out said chromatography is CaptoBlue-Sepharose matrix. Optionally, an extra HPLC purification step can be added using a C4 column.

As an alternative to purification by dye pseudo-affinity chromatography, said purification step comprises a tangential flow filtration step and a subsequent reCG concentration step.

Through the method of production of a recombinant equine chorionic gonadotropin hormone (reCG) of the present invention, it is possible to obtain a reCG with a specific activity (as in vivo potency units in relation to the protein mass determined by ELISA) of at least 6000 IU/mg.

Another object of the present invention comprises a nucleic acid encoding the reCG alpha subunit obtainable by the methods described in the present invention. Said alpha subunit of said reCG comprises a sequence substantially similar to SEQ ID No. 1.

Another object of the present invention comprises a nucleic acid encoding the reCG beta alpha subunit obtainable by the methods described in the present invention. Said beta subunit of said reCG comprises a sequence substantially similar to SEQ ID No. 2.

In a preferred embodiment of present invention, said sequence substantially similar to SEQ ID No 1 means that said sequence has at least 90% identity to a sequence of SEQ ID No 1.

In a preferred embodiment of present invention, said sequence substantially similar to SEQ ID No 1 means that said sequence has at least 95% identity to a sequence of SEQ ID No 1.

In a preferred embodiment of present invention, said sequence substantially similar to SEQ ID No 1 means that said sequence has at least 98% identity to a sequence of SEQ ID No 1.

In a preferred embodiment of present invention, said sequence substantially similar to SEQ ID No 2 means that said sequence has at least 90% identity to a sequence of SEQ ID No 1.

In a preferred embodiment of present invention, said sequence substantially similar to SEQ ID No 2 means that said sequence has at least 95% identity to a sequence of SEQ ID No 1.

In a preferred embodiment of present invention, said sequence substantially similar to SEQ 25 ID No 2 means that said sequence has at least 98% identity to a sequence of SEQ ID No 1.

The percentage identity is calculated by dividing the number of matched portions in the comparison window by the total number of positions in the comparison window, and multiplying by 100. Identity is performed using the BLAST and BLAST 2.0 algorithms (see, e.g., Altschul et al., 1990, J. Mol. Biol. 215:403-410 and Altschul et al., 1997, Nucleic Acids 30 Res. 25(17):3389-3402).

Another object of the present invention comprises a reCG hormone obtainable by the method of producing a recombinant equine chorionic gonadotropin hormone (reCG) of the present invention. Said reCG hormone comprises a glycosylation profile with at least 3% neutral structures, and at least 3% tetra-sialylated structures. Preferably said hormone comprises a glycosylation profile with at least 3% neutral structures, between 26 and 30% mono-sialylated structures, between 50 and 55% bi-sialylated structures, between 8 and 15% tri-sialylated structures and at least 3% tetra-sialylated structures.

Another object of the present invention comprises a pharmaceutical formulation characterized in that it includes a therapeutically effective amount of the reCG of the present invention. In an embodiment aspect, said formulation is lyophilized. In another embodiment, said pharmaceutical formulation is liquid. The pharmaceutical formulation of the present invention further comprises a sugar, a preservative, an antioxidant, mannitol, and anti-aggregation agent. Said liquid pharmaceutical formulation further comprises trisodium citrate dihydrate, citric acid monohydrate, arginine, sucrose, mannitol, L-methionine, poloxamer 188, m-cresol and water.

Another object of the present invention comprises a method for inducing ovulation in animals involving the administration of at least 140 IU/animal of the reCG obtainable by the method of production of a recombinant equine chorionic gonadotropin hormone (reCG) of the present invention. The present method manages to induce ovulation 48 hours after its application. The present invention, for the production of reCG, solves all the disadvantages associated with the use of the hormone obtained from the blood of pregnant mares (PMSG) and those presented by all the recombinant options described so far, none of them having yet reached the veterinary market:
the process for producing the reCG described in the present invention replaces the use of animals to obtain the hormone, eliminating the cruel practices to which they are subjected, which completely go against the bioethical and safety standards that society demands;
the reCG obtained and described in the present invention is a higher quality product, derived from the cultivation of animal cells in bioreactors in serum-free medium that allows the standardization of the production processes. Thus, both the culture parameters and the purification procedures are easily controllable and reproducible (unlike when using an animal host), yielding a product (reCG) of greater consistency between batches, free of contaminants from animal plasma, and therefore safer from a sanitary point of view;
the method described in the present invention allows to achieve a reCG hormone that exhibits in vivo FSH/LH activity, unlike the rest of the technologies described in the literature that use other types of cellular hosts that do not allow to obtain a recombinant hormone displaying in vivo activity;
a less quantity of International Units for the reCG obtained and described in the present invention is needed to treat the animals. Since the reCG of the present invention is more effective, only 100 ILi/animal for Bos indicus and 140 ILi/animal for Bos taurus are required for IATF instead of the 300 IU and 400 IU, respectively, which are required when working with the PMSG obtained from pregnant mares. Also, just 1000 IU are needed for Bos indicus and 2000 IU for Bos taurus for SOV, instead of the 2000 IU and 4000 IU that are needed, respectively, when working with the PMSG obtained from mares;
after treatment with 140 IU of reCG per animal, the cyclicity in anestrus cattle is restored in the 85% of the cases, in comparison with a 65% of success after PMSG treatment. This is very important since these animals with very low body condition only enter a state of cyclicity due to the action of the hormone on the ovaries. If the hormone is not present, the animal remains in an acyclic state (in anestrus) and, therefore, represents a non-productive animal;
a more effective synchronization of animals is achieved. This significantly facilitates their use in fixed-time artificial insemination (FTAI) and superovulation (SOV) protocols. All cows treated with the reCG of the present invention for FTAI ovulate 48 h after administration, whereas PMSG-treated cows ovulate between 50 and 60 h after administration. Having a fixed time for the insemination-pregnancy procedure to be effective and profitable is essential for livestock management, where hundreds of cows are treated. In this way, cows treated “in the morning” can be inseminated at the same time two days later;
compared to other PMSG substitutes obtained in animal cells, for example CHO DG44, which seem to exhibit adequate glycosylation profiles, the present invention allows obtaining a reCG with the highest productivity described to date. In this way, the present invention overcomes the main limiting disadvantage that has prevented the existence of a recombinant version of the hormone on the market: the present invention is the first technology able to generate large amounts of reCG with biological activity at low cost. The high productivity together with the use of a low cost serum-free culture medium contributes to the decrease of the overall cost of the method of the present invention. Both the reCG obtained using a high recovery single-step purification method or the resulting from a single tangential flow filtration and concentration procedure of the initial sample, exhibit adequate glycosylation profiles and, therefore, show in vivo biological activity in rats, cows and pigs;
a liquid formulation of reCG is achieved which is stable over time and thus superior to the existing marketed products, that consist of lyophilized or frozen liquid formulations, with all the disadvantages that these entail.

Coming up next, examples of the assays performed to obtain and carry out every object of the present invention are described. It should be noted that these examples are by way of illustration without the intention of restricting the protecting spectrum of the present invention.

### EXAMPLES

### Example 1—Coding Sequence Design and Optimization

The technology developed in the present invention first involved the development of mammalian cell lines, in particular, suspension CHO-K1 cells (Chinese Hamster Ovary cells), which produce recombinant equine chorionic gonadotropin (reCG). The coding sequences of the a and 13 subunits of reCG (reCGa and reCGl3) were optimized for expression in CHO-K1 cells, to obtain high levels of mRNA and thus maximize the expression of the encoded protein. Gene optimization takes advantage of the degeneracy of the genetic code, whereby a protein can be encoded by various alternative genetic sequences. Because codon usage differs in each organism, this can create drawbacks in expressing recombinant proteins in heterologous hosts, resulting in very low expression. Thus, gene optimization algorithms allow multiparameter optimization of DNA sequences, encompassing multiple aspects of gene expression: transcription, splicing, translation and degradation of mRNA, to achieve the most efficient expression of a given protein.

After optimization, it was observed that the recombinant sequence of the eCGbeta showed an 82.2% homology with the sequence of the non-optimized beta subunit (“natural” eCGbeta), while with other recombinant sequences mentioned in the background of the invention section, homology was lower (78%). It was observed that those nucleotide positions of the coding sequences on the optimized recombinant hormone of the present invention that differed both with the residues of the native sequence and with those of the optimized sequences published in the state of the art corresponded to 4% of the total, which was sufficient to be a determining factor in obtaining higher levels of reCG expression with respect to those reported in other patents.

The synthetic sequences were obtained as DNA and then cloned into p-alpha_eCG (AmpR) and p-beta_eCG (AmpR) vectors, respectively. These vectors contain a bacterial replication origin (Col E1 origin), which allows plasmid amplification into E. coli and an ampicillin antibiotic resistance gene (AmpR). The vector p-eCGa has 3000 bp and contains the 360 bp eCGa coding sequence, where the first 72 bp encode for the natural eCGa signal peptide. The p-eCGl3 vector contains 3200 bp and includes the 507 bp eCGl3 coding sequence comprising a signal peptide of 60 bp long at the start of this sequence.

### Example 2—Expression Vectors Construction

To obtain stable reCG-producing cell lines, third-generation lentiviral vectors were constructed as a genetic material delivery method. For this purpose, first it was necessary to clone the coding sequences of each subunit in the lentiviral transfer vectors, and then carry out the assembly of lentiviral particles (LP) and their subsequent titration.

Lentiviral expression vectors encoding the a and 13 subunits of reCG were constructed. Thus, the vector containing the a subunit was digested with the XbaI/EcoRV enzymes to release the reCGa coding sequence, which was cloned into the NheI/EcoRV sites of the lentiviral plasmid vector pLVenhCEF. The vector containing the 13 subunit was digested with the BamHI/EcoRV enzymes to release the reCGl3 coding sequence, which was cloned into the BamHI/SmaI sites of the pLVenhCEF vector. This vector, developed in our laboratory, includes the EF-1a promoter as an expression regulatory element, characterized by high expression levels in a wide variety of animal cells. It also includes an expression-stimulating fragment derived from a CMV enhancer sequence and a coding region for a puromycin resistance gene as a selection marker. The generated plasmids were amplified by prokaryotic cells (E. coli), cultured in shaking conditions and purified by organic solvent extraction and precipitation. Sequencing of the selected E. coli clones confirmed the identity of the DNA fragments cloned in the plasmids pLVenhCEF-reCGa and pLVenhCEF-reCGl3, demonstrating 100% homology to the sequences of the synthetic eCGa and eCGl3 genes, respectively.

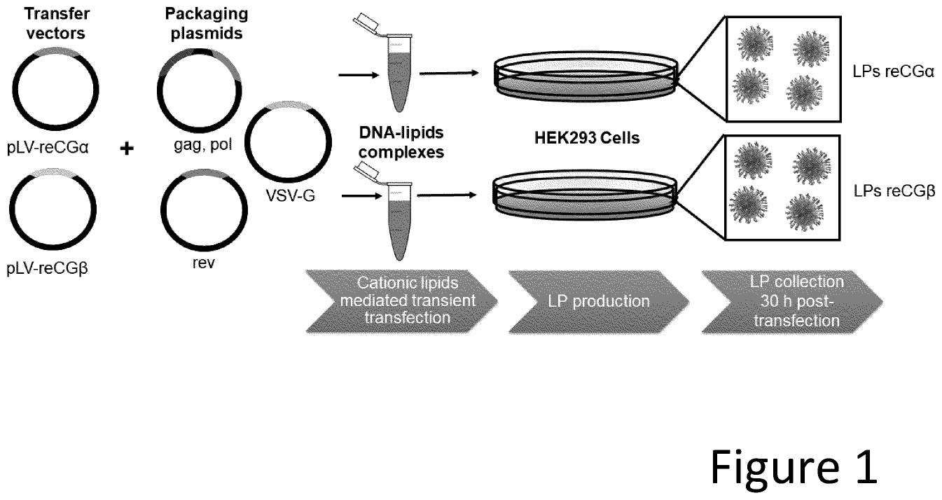

To produced third-generation lentiviral particles, transient transfections of HEK293 cells (packaging cells) were carried out with four plasmids: pREV, pGlyco-G, pMDL (packaging plasmids) and the transfer vectors pLVenhCEF-reCGa and pLVenhCEF-reCGl3 (encoding each reCGsubunit). For this purpose, cationic lipids were used as DNA carrier. Supernatants containing the lentiviral particles were harvested 30 h post-transfection, centrifuged at 65.000 g for concentration and stored at 80° C. until use (FIG. 1).

Lentiviral particle titration was performed using the QuickTiter™ Lentivirus Titer Kit (Cell Biolabs Inc.). This kit was designed to detect the lentivirus associated HIV-1 p24 core protein only, therefore, the free protein that remains in supernatant does not interfere with the assay. Thus, a physical titer of 2.7×109 LP/ml and 2.1×109 LP/ml was obtained for reCGa and reCGl3, respectively, which can be approximated to a transduction titer of 5.0×106 and 3.9×106 TU/ml, respectively, resulting in high titers for performing transductions of CHO-K1 cells.

### Example 3—Cell Lines Generation

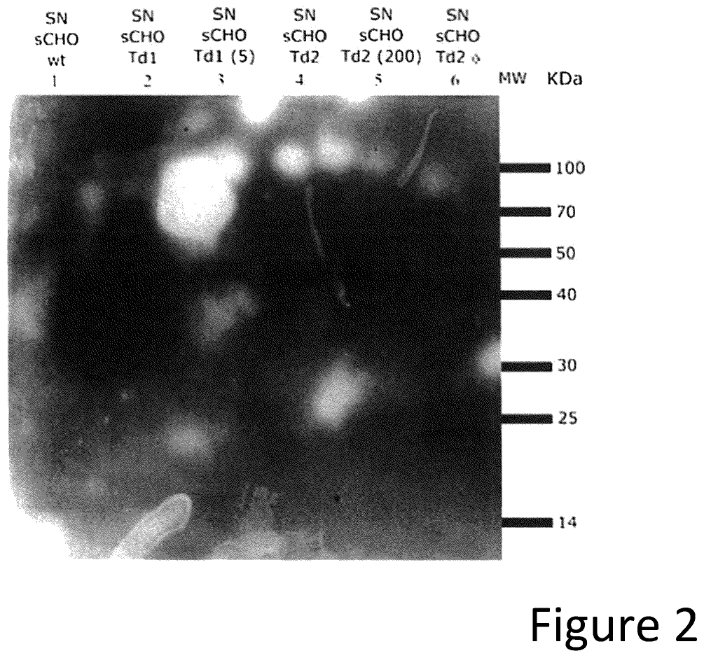

The obtained lentiviral particles were used to generate reCG-producing recombinant CHO-K1 cell lines in suspension. A total of two successive transductions events (Td1 and Td2) were performed, since cells did not survive to a third transduction event. The transduced cell lines were subjected to selective pressure by incubation with increasing concentrations of puromycin. This strategy allowed the population to become enriched with cells resistant to higher concentrations of the antibiotic, thus leading to an increment of the productivity of the whole cell line. FIG. 2 shows an SOS-PAGE assay followed by western blot using specific polyclonal anti-reCG antibodies. As it can be observed, for the same cell density, the highest reCG concentration was obtained in the supernatant of the sCHO Td2 (200) cell line (which was resistant up to 200 μg of puromycin).

Subsequently, reCG productivity of the generated cell lines was evaluated by determining the accumulated hormone concentration in the supernatant and the initial and final cell density after a certain period. The reCG quantification was performed using a competitive ELISA developed in our laboratory. The assay involved competition between the solid phase-immobilized antigen (reCG) with the same antigen (reference reCG or unknown sample) in solution for binding to specific rabbit anti-reCG antibodies (pAb anti-reCG). These antibodies were previously obtained in our laboratory. Subsequently, peroxidase-conjugated secondary antibodies were added to detect the remaining solid phase-bound complexes. The productivity of the different cell lines is summarized in Table 1.

TABLE 1
Specific reCG productivity of generated cell
lines at laboratory-scale

| | Productivity |
|---|---|
| Cell line | (μg × 10⁶ cens-¹ × d-1) |
| sCHO Td1 | 0.37 |
| sCHO Td1 (5) | 0.32 |
| sCHO Td2 | 0.96 |
| sCHO Td2 (200) | 0.78 |

Considering these results, the highest reCG producing cell line (reCG Td2 cell line) was selected to be cloned.

### Example 4—Clonal Isolation

The reCG productivity of the generated cell lines allowed selecting one of them, obtaining a single-cell clone with adequate growth profile and high reCG productivity.

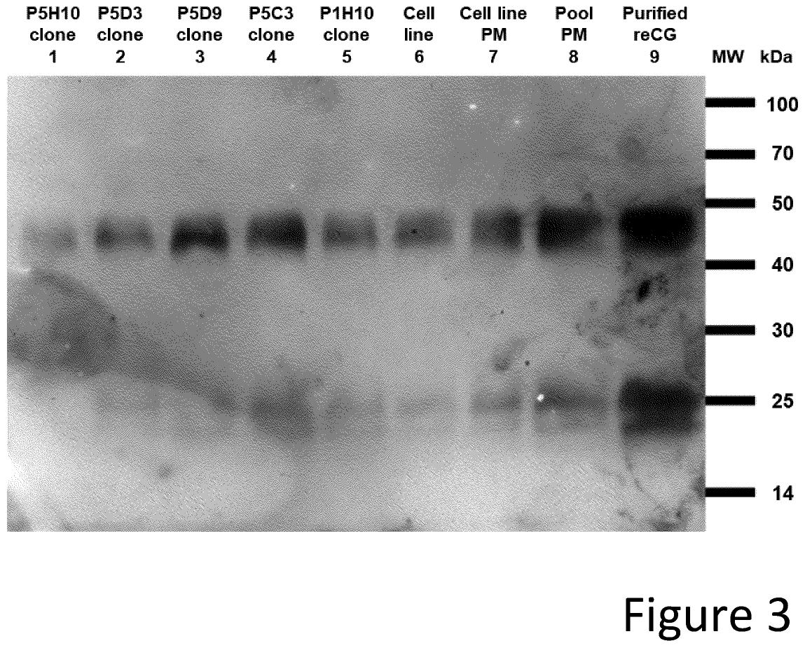

To select the clones with the highest reCG expression level, more than 400 clones were evaluated in an initial dot-blot screening, using specific anti-reCG antibodies. The selected clones were cryopreserved. After a preselection step to reduce the number of clones to be analyzed, the “apparent” productivity (determined as the concentration of reCG obtained for the same cell density for each clone) of the selected clones was evaluated by SOS-PAGE followed by western blot (FIG. 3). This analysis revealed that clones P5D9 (lane 3) and P5C3 (lane 4) exhibited the highest reCG expression level. Subsequently, reCG productivity of the selected clones was evaluated by determining the accumulated hormone concentration in the supernatant and the initial and final cell density after a certain period. The reCG concentration was determined by the competitive ELISA described above. The estimated productivity was 0.80 and 0.81 μg×10⁶ cells-¹×d-¹ for P5D9 and P5C3 clones, respectively. Finally, P5C3 clone was selected since it presented better growth performance than P5D9 clone.

### Example 5—Optimization of Culture Conditions to Achieve Optimal Glycosylation

5.1 High Density Culture of PSC3 Clone in Serum-Free Medium in One-Liter Bioreactor

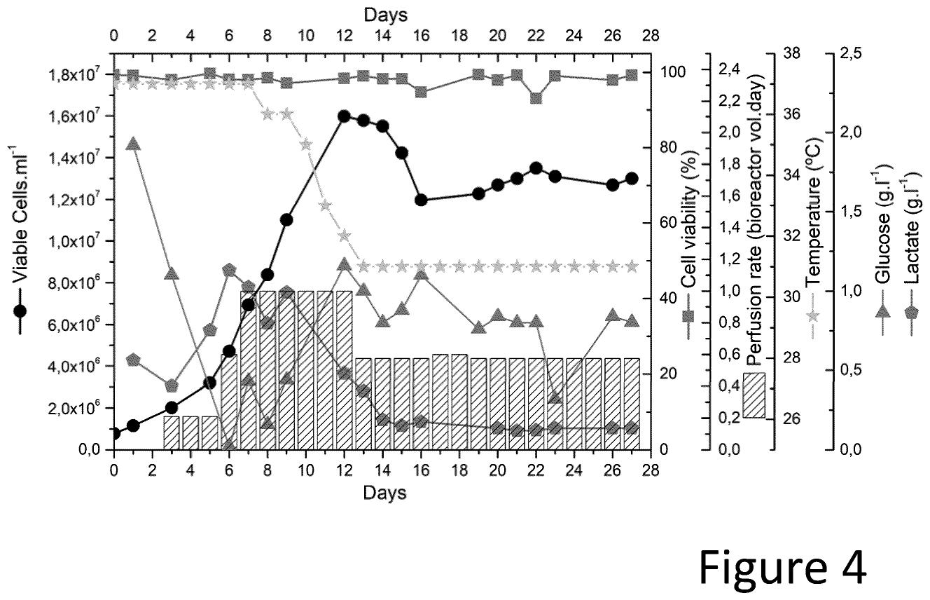

P5C3 clone was cultured in fetal bovine serum-free culture medium (MC01) in a one-liter bioreactor in perfusion mode for 27 days. The culture was started at a cell density of 7.8×10⁵ cell·mL-¹ and showed exponential growth, with no lag phase, until reaching a maximum cell density of 1.6×10⁷ cell·mL-¹. Cell viability was above 94%. The perfusion rate was started on day three of culture and it was varied between 0.21 and 1.00 reactor volumes per day. Lactate concentration remained below 1.1 g·L-¹ (12.2 mM) (FIG. 4). The specific growth rate of the clone was 0.013 h-¹.

Afterwards, a new production medium (MC02) was formulated. Important: the composition of this culture medium arises from combining a commercial medium with salts, amino acids, carbohydrates, etc. (Table 2). This was performed to optimize the culture medium cost, 10 reducing its value by 50%.

This new composition did not alter cell the productivity nor any characteristic of the molecule.

TABLE 2
Culture medium (MC02) composition

| | g.1-1 |
|---|---|
| Excell 302 | 10.5 |
| Bicarbonate | 1.6 |
| Glutamine | 0.5825 |
| Glucose | 2.25 |
| NaCl | 4.41 |
| KH2PO4 | 0.304 |
| Na2HPO4 | 0.32 |
| Poloxamer 188 | 0.5 |

5.2 High Density Culture of PSC3 Clone in Serum-Free Medium in SO-Liter Bioreactor

Culture at production scale. After culturing the cells in one-liter bioreactor, scaling to 50-liter bioreactor was performed, reproducing the cell culture parameters tested on the laboratory scale. Since the perfusion rate was one reactor volume per day, 50 L of harvest was obtained in this fermentation process, each of them containing 157 IU/ml of reCG. Considering that one dose of reCG of the present invention consists of 140 IU, and that 50,000 ml of supernatant containing 157 IU/mll were harvested, a total of 7,850,000 IU was obtained in 24 h. Hence, the production method of the present invention generates around 56,000 doses of reCG per day at production scale (50 L). Thereby, more than one dose/mi of harvest is obtained at production scale. Comparing these results with the current production method (extraction from pregnant mares), the culture conditions developed in a 50 L bioreactor represent about 600 mares. In other words, the technology reported in the present invention could produce in a 25-30 days bioprocess (including culture, purification, formulation, packaging) the same number of doses than 600 pregnant mares in 200 days [assuming that all mares have the same concentration of eCG in their blood and that the eCG obtained from each mare has the same quality (which is impossible)].

Furthermore, reCG productivity of P5C3 clone on a production scale (50 L bioreactor) was calculated in different culture media bearing different costs (Table 3). These culture media included the original commercial medium (MC01, EX-CELL 302), the optimized MC02 medium (previously described) and the MC05 medium. In this last medium, an even higher productivity was obtained, making a greater difference with respect to results published by other authors, and also at a lower cost.

TABLE 3
reCG productivity of P5C3 clone in different media in
50 l bioreactor and cost of each culture medium.

| | Productivity | |
|---|---|---|
| | (Ul × 106 | MC Price |
| Culture medium | ceU-1 × d-1 | (U$S/I) |
| MC01 (original commercial medium) | 15.74 | 30.80 |
| MC02 (optimized medium) | 16.83 | 15.97 |
| MCOS (commercial medium) | 29.71 | 5.00 |

Culture Medium 01 (MC01): Ex-Cell CHO 302

Culture Medium 02 (MC02): MP01/P2G 50/50 (MP 02) optimized medium Culture

Medium 05 (MC05): BHK-21 CD Production Medium

In patent application WO2017112987 they use the DHFR-MTX gene amplification system. They do not report productivities, but instead report kinetics of reCGl3a expression after adaptation to growth in the absence of MTX. From reading the document, approximately a cumulative value in IU/mL of reCG is obtained for the cell line cultured in the presence of fetal bovine serum of 10 IU/ml (24 h), 20 IU/ml (48 h) and 28 IU/ml (72 h), and for the line cultured in the absence of fetal bovine serum of 5 IU/ml (24 h), 10 IU/ml (48 h) and 18 IU/ml (72 h).

Compared to those results, the cell clones obtained in the present invention produce 45.6 IU/ml (P5D9) and 50.2 IU/ml (P5C3) at small scale, in the absence of fetal bovine serum, in 72 h, while in continuous perfusion mode in bioreactors, productivity levels greater than 15 IU×10⁶ ceU-¹x d-¹ are reached. This corresponds to 157 IU/ml in 24 h, representing a significantly higher value than the ones reported in patent WO2017112987 (they reported 5 IU/ml in the absence of fetal bovine serum in 24 h). This means a production of more than 7,000,000 IU per day, representing more than 50,000 doses per day of reCG of the present invention.

In this way, the technology of the present invention allowed obtaining the highest productivity and production values reported to date, which resulted from a conjunction of unique factors of our technology: the optimization of sequences for expression in CHO-K1 cells (Cricetulus griseus species), the use of third-generation lentiviral vectors of our own property, and the use of the CHO-K1 cell line, which expresses a wide range of glycosyltransferases, capable of adding bi-, tri- and tetra-sialylated complex-type N-glycans to polypeptides, in addition to generating mono- and bi-sialylated mucin-type O-glycans, which are key factors for eCG to exhibit in vivo bioactivity.

Indeed, the exclusive DNA sequence optimization process was effective to achieve high hormone expression, as evidenced by the 4% of nucleotides that have been modified in the protein's native DNA sequence and that they have not been altered in the synthetic sequences reported by other patents.

### Example 6—Purification

6.1—First Purification Step

After CHO cell culture in a bioreactor in Excell 302 (Sigma) serum-free medium, the harvest material was employed to develop a first capture purification step. Thus, a dye-pseudoaffinity chromatography was selected, employing a CaptoBlue-Sepharose resin packed in a XK column (GE, Healthcare) and equilibrated in 20 mM Tris-HCl buffer pH 7.

The clarified harvest without previous conditioning was loaded onto the resin using a flow rate of 153.06 cm/hand a total retention time of five min. After a washing step with the same equilibration solution, the protein was eluted using an isocratic gradient (Tris-HCl buffer pH 8, 2 M NaCl, 20% (v/v) ethanol). No leakage of the hormone was observed during the loading and washing steps of this first chromatography, thus, the loaded conditions were adequate. The intact hormone was recovered with a notably higher purity level than that obtain from partial purification from pregnant mare's serum (PMSG). Thus, the reCG capture step from cell culture supernatant was optimized, employing a dye-pseudoaffinity resin. A high yield was achieved without any loss of protein, since the recovery was 98% (evaluated by both ELISA and RP-HPLC).

6.2—Second Purification Step

Hydrophobic interaction chromatography was selected as second purification step, since partially purified hormone from the first capture step (named post-Blue) was eluted under high ionic strength conditions (2 M NaCl). To reduce de number of operation units and the cost of the global purification process, the following strategies were proposed: 1) to load the “crude eluate”, i.e. no preconditioned post-Blue fraction, avoiding a diafiltration step (since this fraction is in a high ionic strength condition); 2) to screen two type of hydrophobic ligands, both available in our laboratory: Phenyl and Butyl; 3) to evaluate the eluate diafiltered against citric/citrate buffer pH 6.0, as this is the condition in which the post-Blue API (named FD1RECG) is formulated and 4) to evaluate the purification performance employing different salts: first, NaCl (because it is the one present in the post-Blue buffer), then, Na2SO4, and finally, (NH4)2SO4 (as this is the salt with the highest hydrophobic effect). Considering the purification performance (recovery and purity degree) of all strategies, the best conditions were as follows: the DF1REG was loaded onto a Butyl Sepharose 4FF resin under a flow rate of 15 cm/hand a total retention time of three min. To improve the hydrophobic interaction between the protein and the ligand, the resin was equilibrated with 50 mM citric/citrate buffer pH 6.0, 2 M (NH4)₂SO₄ and the sample (DF1REG) was conditioned employing the same equilibration buffer. Thereafter, two washing steps were performed: the first using the same buffer as the one employed in the equilibration step and the second with a lower ionic strength (50 mM citric/citrate buffer pH 6.0, 1.5 M (NH4)2SO4) to remove impurities. Finally, an isocratic elution was performed, employing 50 mM citric/citrate buffer pH 6.0, 0.5 M (NH4)2SO4_

### Example 7—Formulation

7.1—Liquid Formulation of the Final Product

7.1.1—Development of reCG Liquid Formulation Applying QbD Tools

Through the present invention, a reCG liquid formulation that allowed obtaining a stable and, thus, active hormone in liquid form was developed. In this way, the lyophilization procedure, which represents a more exhaustive and higher cost unit operation could be avoided. Hence, obtaining a liquid formulation instead of a lyophilized one, guarantees not only lower costs, but also a reduction in production process cycle, avoiding the reconstitution step of the lyophilized product. Besides, in case of working with a multi-dose presentation, the required amount can be fractioned, ensuring its prolonged stability during use.

7.1.1.1—Preformulation Assays-Establishing the Significant Factors Influencing reCG Stability in a Liquid Formulation

For thermal forced degradation studies of reCG purified by CaptoBlue-Sepharose chromatography, the temperature was modified between 20 and 70° C., employing a pH range between 3.0 and 8.0. The samples were heated for ten min at each condition in a thermocycler and stored at −70° C. until analysis. Then, an aliquot of reCG in each condition was evaluated in a non-reducing SOS-PAGE following by a Coomassie Brilliant blue staining to visualize the reCG dissociation degree. Differences in the mobility profiles in SOS-PAGE indicated that the pH had an impact upon the reCG heterodimer stability. Samples corresponding to the low pH range (pH 3.0 to 5.0) incubated at the high temperatures showed a different pattern of bands compared to those corresponding to the more basic pH range (pH 6.0 to 8.0). In parallel, the emission (fluorescence) profile was evaluated (A excitation: 274 nm) to analyze possible conformational changes. A red-shift in those samples subjected to higher temperatures and lower pH values was observed. A red shift could be correlated with a higher denaturation protein pattern or a loss of native conformation. Taking into 25 account these results, and with the aim of reducing the chemical degradation process (mainly, deamination and oxidation), which have lesser effect at neutral pH, the optimum pH range was established between 5 and 7. Likewise, as the pl (isoelectric point) of the protein was close to 3.5 to 5.5 (according to the IEF assay), working in a pH range between 5 and 7 will guarantee that the protein exhibits a negative net charge, reducing physic degradation events as aggregation (contrary to what might occur at pH values close to the reCG's pl).

7.1.1.2—Formulation Assays

One of the major challenges in biotherapeutic proteins production process is to achieve a formulation that guarantees high protein quality and stability. By combining design of experiments (DoE) with simple analytical techniques and accelerated stability assays, a liquid formulation was achieved, allowing to maintain a 98% of the biological potency of reCG (intact, active reCG) for up to six months under accelerated conditions (25° C., 60% relative humidity, RH) (CAMEVET, 2012).

A Placket-Burman design (PBD) was employed to determine the effect of several factors on the reCG stability under accelerated conditions (25° C., 60% RH, seven days). Twelve experiments were performed with triplicates in the central point to study the standard deviation of the effects in N=15 (number of experiments). The effect of eight real factors and three dummy variables (virtual variables) on reCG stability was evaluated. The analyzed factors were: amount of stabilizers (sucrose, mannitol, Arg, L-met) and surfactant (Poloxamer 188), molarity and pH of the buffer, and concentration of the API (reCG dose). The response consisted in analyzing the reCG amount (%) after seven days of storing at 25° C., 60% RH by determining the area under the curve of the intact reCG assessed by RP-HPLC technique (tR: 13.58 min).

The Pareto chart was employed to determine the influencing factors. Thereafter, an ANOVA test was applied to study the factors' effects on the response and to confirm the significant effect of the factors. The obtained model satisfied the assumptions of normality, homoscedasticity and independence of the variables. Data analysis indicated that the significant factors on the reCG stability under accelerated conditions were buffer molarity (p: 0.0013), L-met (p: 0.0171), sucrose (p: 0.0044) and surfactant (p: 0.0097) amount. Further, the R-squared (0.8929) and the adjusted R-squared (0.8393) indicated a good relationship between the experimental data and the fitted data.

During the optimization stage of the reCG liquid formulation, a four-factor (buffer molarity, amount of sucrose, L-met and Pluronic F-68), five-level Central Composite Design (CCD) was performed. Twenty-seven runs were carried out and the response consisted, once more, in analyzing the reCG amount (%) after 0, 6, 12, 60, 90, 125 and 150 days of storing at 25° C., 60% RH and 40° C., 75% RH by RP-HPLC. The analyzed critical factors were buffer molarity, sucrose, surfactant and antioxidant amount. The factors that were found to have no significant effect on reCG stability were kept at constant concentration levels in all formulation tests.

After 90 days, significant differences between formulations were observed. Thus, the reCG stability in the 27 formulations were fitted to quadratic model. The obtained hierarchical model satisfied the assumptions of normality, homoscedasticity and independence of variables. Furthermore, the adjusted R-squared (0.7238) indicated a good relationship between the experimental data and the fitted data: Raf 0.8122; R²a (0.7238; CV % 1.53, lack-of-fit: 0.1107 (p<0.05 is significant).

A robust design space was obtained, and an optimum formulation condition was selected, consisting of 70 mM citric/citrate buffer (pH 6.0), 161 mM sucrose, 1.0 mg/ml I-met and 1.0 mg/ml surfactant (plus 5 mM I-Arg and 5 mg/ml mannitol).

This predicted reCG liquid formulation was validated by preparing three individual formulation batches and evaluating the stability after 0, 15, 45 and 90 days of storing at 4° C., 25° C., 60% RH and 40° C., 75% RH. After 90 days, an intact reCG amount (assessed as auc by RP-HPIC), of 125±3, 125±1 and 94.6±0.2% at 4° C., 25° C./60% HR and 40° C./75% HR, respectively, was obtained, demonstrating the robustness of the liquid formulation developed. The validated liquid formulation was evaluated up to 150 days, obtaining a 98±13% of intact reCG under accelerated conditions (25° C./60% RH).

7.2—Formulation of the Lyophilized Product

A stable solution or formulation is one that assures that the degradation degree, alteration, aggregation or loss of biological activity is acceptable or manageable. Ideally, the formulation must retain at least 80% of the initial potency of the protein during a period of six months stored at 2-8° C. (U.S. Pat. No. 7,740,884 B2). In this way, a solid-state excipient formulation was developed, containing salts as buffering agents such as citric/citrate, at a low molarity (10 mM) and pH 6.5.

Besides, the success of lyophilization in the solid state involves the balance of two competing requirements: the generation of a rugged cake that does not collapse during primary drying, and the existence of an amorphous state that allows the interaction between the excipients and the protein (Jhonson et. al., 2001). Excipients that act as protein stabilizers such as sucrose or trehalose behave like amorphous solids, whereas excipients like mannitol act as crystalline solids that reduce the cake collapse. Thus, in the present invention, a ratio of 4:1 mannitol:sucrose was employed, in concentrations of 40 g/l and 10 g/l, respectively.

Proteins like glycoprotein hormones are susceptible to oxidation degradation; therefore, the use of compounds with antioxidant behavior is desirable, such as some amino acids like methionine, chelating agents (EDTA, for instance) or sodic bisulfite. Besides, to prevent adsorption of reCG on the surface of the vial and reduce protein interaction in air-water interface, a non-ionic surfactant (Pluronic F68 or Poloxamer P188) was employed. In the present invention, 0.1 mg/ml and 0.25 mg/ml of methionine and Poloxamer P188 were employed, respectively. Ideally, the reCG amount in the reconstituted cake must be close to 1,500 IW/ml, being the final volume the same as the initial one (3 ml).

The process consisted of mixing the formulation excipients with the API, filtering using 0.2-mm PES (polyethersulphone) filters, filling appropriately washed and sterilized borosilicate vials, (sealed with rubber stoppers) and lyophilizing according to the state of the art.

### Example 8—Biochemical and Physicochemical Characterization

8.1 Methodology

The reCG was produced by culturing suspension P5C3 clone in a one-liter bioreactor in perfusion mode (Biostat Q Plus, Sartorius) in serum-free medium. Then, the clarified supernatant (Sartobran-P 0.45 μm, Sartorius) was purified employing a CaptoBlue-Sepharose chromatography as a capture step. Afterwards, to obtain aliquots of the protein with higher purity degree, two alternative purification steps were evaluated:
a) Reversed-phase high performance liquid chromatography (RP-HPIC)
b) Hydrophobic interaction chromatography (HIC)

The reCG molecule purified by RP-HPIC was denominated reCG RP-HPIC, whereas the reCG molecule purified by HIC, was named reCG HIC.

Commercial preparations of eCG from Foli-G, Zoovet SA. (Argentina) and Novormon, Syntex (Argentina) were purchased from regional veterinary drugstores and employed as internal reference standards. The codes A and B were assigned to Foli-G and Novormon, respectively.

8.1.1—RP-HPLC for Structural Analysis. RP-HPLC to Assess the Biological Potency

The quali- and quantitative studies were performed employing a C4 column, with gradient elution and UV detection (210 nm).

A good correlation between the potency assay in rats and the intact reCG measured as the auc by RP-HPIC technique was demonstrated, applying an EJCR (elliptical joint confidence region) test. Here the EJCR test and a bilinear least square (BIS) regression method were applied. If the ideal point (1,0) was included in the ellipse, it could be assumed that the method was accurate. This elliptical region is described by mathematical equations that are drawn in bi-dimensional graphics. The ellipse size is related with other analytical parameters as the precision of the assay.

8.1.2—SOS-PAGE

Throughout this assay, the purity and apparent molecular weight was analyzed in non-reducing conditions. A colorimetric detection (Coomasie Brillant Blue) or immunochemical detection (western blot) was performed. For western blot analysis, rabbit polyclonal anti-reCG serum produced in our laboratory was employed.

8.1.3—Isoelectrofocusing (IEF)

To separate the protein variants isoforms, an IEF was performed, employing a Pharmacia® equipment composed of an electrophoresis tank (Multiphor II), cooling bath (Multitemp III) and a voltage source (EPS3500XL). The pH range was established using 75% (w/v) 3-5 ampholytes and 25% (w/v) 5-7 ampholytes (GE Healthcare). The detection was performed by Coomasie blue colloidal staining or western blot analysis.

8.1.4—Size Exclusion Chromatography (SEC)-HPLC

The purity and identity of the reCG variants and PMSG were determined by size exclusion chromatography (SEC)-HPLC carried out on a TSKgel G3000SW with a particle size of 10 μm and UV detection.

8.1.5—Spectrofluorometric Analysis

Spectrofluorometric measurements were performed using a Perkin-Elmer LS-55 luminescence spectrometer equipped with a Xenon discharge lamp, Monk-Gillieson type monochromators and a gated photomultiplier connected to an AMO Sempron PC using Windows Xp.

8.1.6—High-pH Anion-Exchange Chromatography with Pulsed Amperometric Detection (HPAEC-PAD)

Sialic acid content was determined by acid hydrolysis of the samples followed by high-pH anion-exchange chromatography with pulsed amperometric detection (HPAEC-PAD) using a DIONEX ICS-5000 system equipped with a CarboPac™ PA20 column (Thermo Fisher Scientific Dionex). N-acetylneuraminic (Neu5Ac) acid standard (Calbiochem, France) was employed as reference standard.

Besides, type and amount of neutral monosaccharides present in purified reCG and PMSG glycans were determined by acid hydrolysis of the samples followed by HPAEC-PAD using a DIONEX ICS-5000 system equipped with a CarboPac™ PA20 column. Monosaccharide mix standard solutions (CM-Mono-Mix-10, Ludger, UK) were treated like sample solutions and used for the identification and quantification of peaks deriving from glycoproteins samples.

8.1.7—N-Glycan Analysis

8.1.7.1—Enzymatic N-Deglycosylation Under Denaturing Conditions

To remove N-glycans from purified samples, enzymatic digestion was performed under denaturing conditions, employing PNGAse F kit (Biolabs Inc.).

8.1.7.2—Weak Anion Exchange Chromatography (WAX) for Charged Labeled N-Glycans Analysis

Released N-glycans were purified by ethanol precipitation and labeled with 2-AB fluorophore. Finally, to analyze the relative amount of neutral, mono-, bi-, tri- and tetrasialylated structures of the proteins, weak anion exchange (WAX) chromatography was performed.

8.2—Results

8.2.1—Sample Preparation

Clarified cell culture supernatant of P5C3 producer cell clone cultured in one-liter bioreactor (perfusion mode), was purified employing a dye-pseudoaffinity chromatography (CaptoBlue-Sepharose, GE, Healthcare) as a first capture step. Thereafter, two alternative chromatography steps were performed: RP-HPLC or HIC (as it was previously indicated).

Biochemically and physicochemical characterization were performed in comparison with PMSG preparations of two commercial branches.

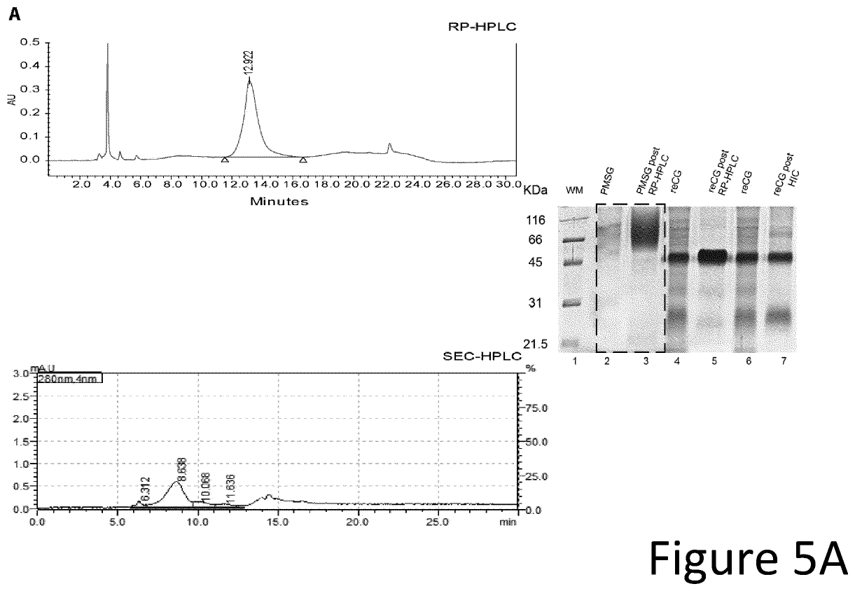

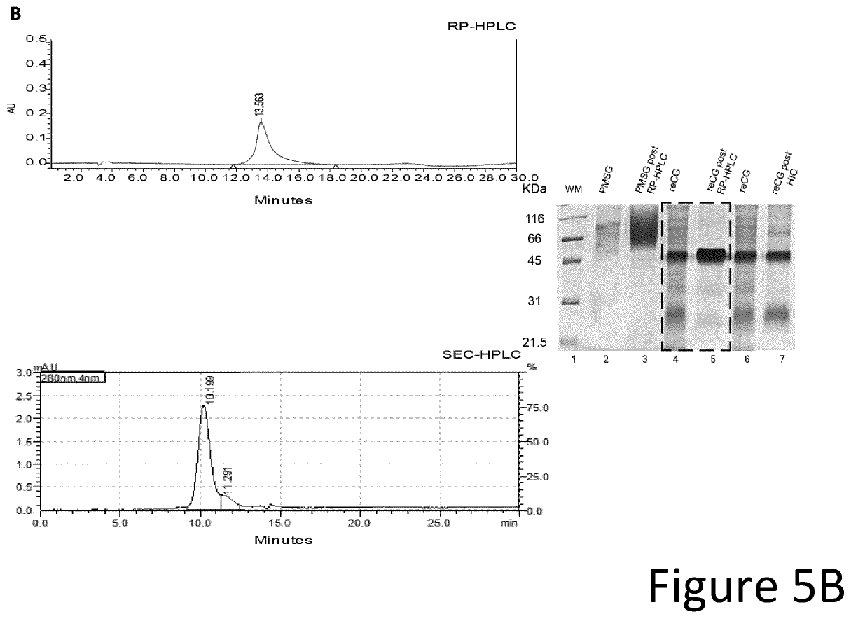

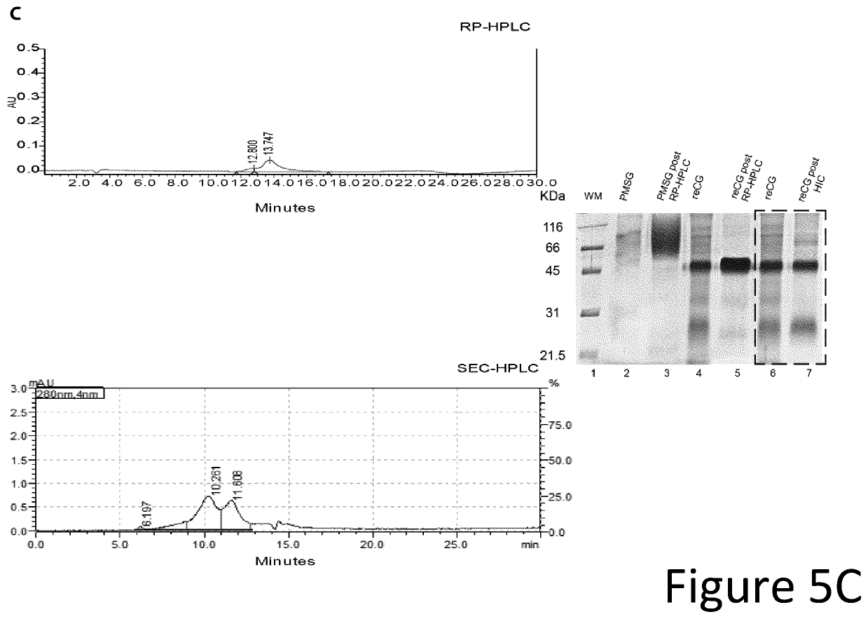

Throughout SEC-HPLC, the purity degree of the different molecules was analyzed. A 90% purity was obtained for reCG RP-HPLC, the remaining percentage probably corresponding to a and 13 subunits of the heterodimer that dissociate during the RP-HPLC purification methods. By the contrary, the reCG molecule purified by HIC presented a 55% of purity, being the main impurity an excess of free alpha subunit (43%). The PMSG preparation purified by RP-HPLC showed a purity of 73% (FIG. 5).

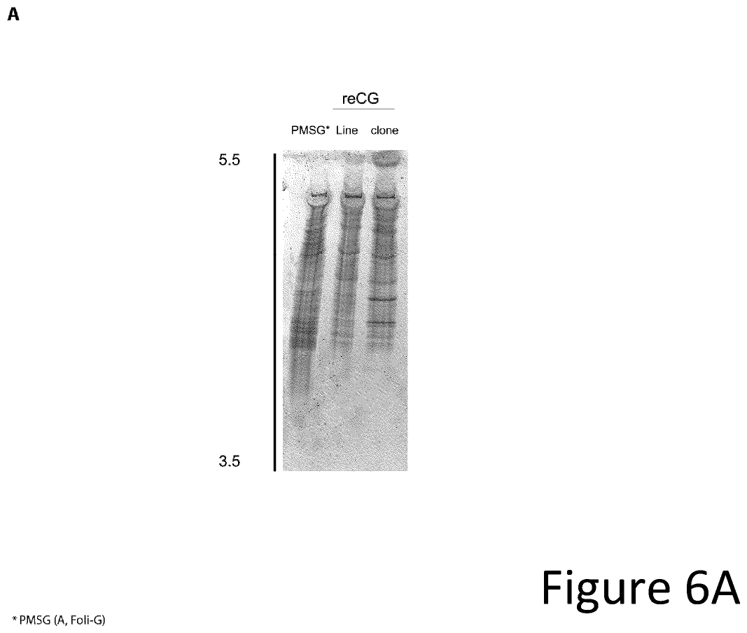

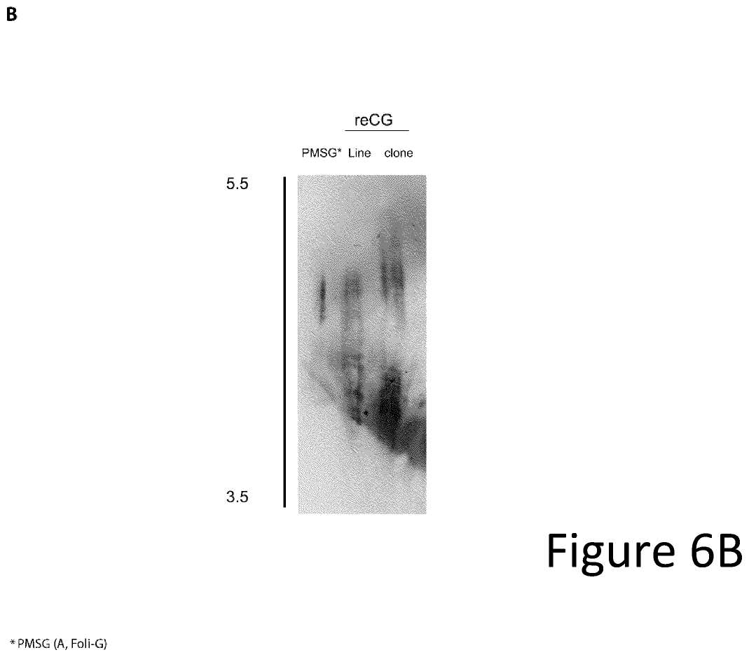

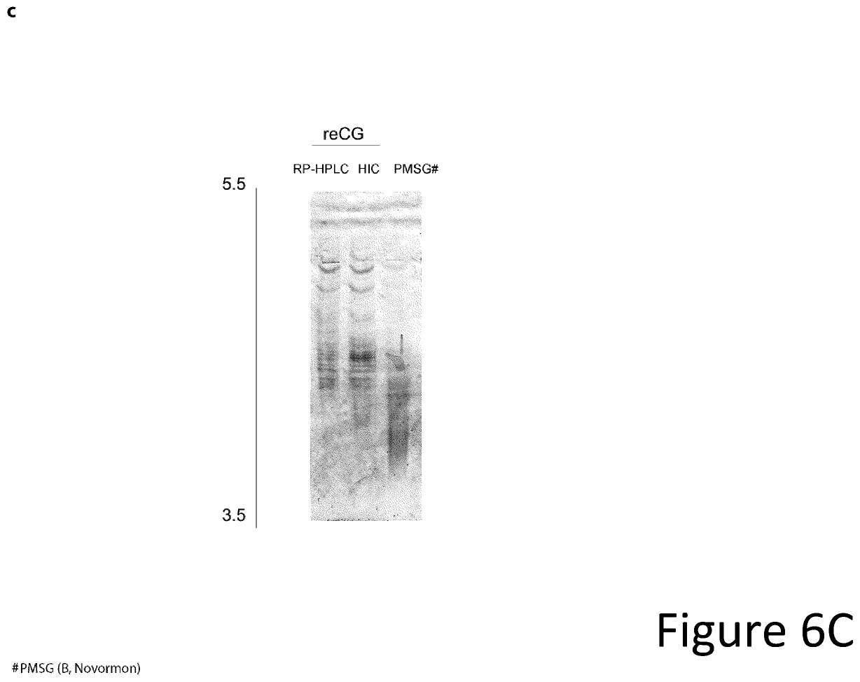

Throughout SOS-PAGE, SEC-HPLC (FIG. 5) and IEF (FIG. 6) it was possible to determine the apparent molecular weight and the isoform profile of the different preparations. The molecular weight of the commercial PMSG was approximately 67 and 66 KOa, analyzed by SOS-PAGE and SEC-HPLC, respectively; whereas the recombinant variant presented a molecular weight of 45 KOa, and 46 KOa, analyzed by SOS-PAGE and SEC-HPLC, respectively. Both recombinant and commercial PMSG preparations exhibited complex isoelectric focusing patterns, with a wide diversity of glycoforms of low isoelectric point. However, although both hormones shared a considerable number of isoforms, PMSG showed a higher proportion of glycoforms concentrated in the more acidic zone of the pH (being this effect more marked for Novormon), whereas recombinant variants presented a wider spectrum of isoforms distributed all over the pH range.

The purified PMSG preparation presented a tR equal to 12.954 min, whereas purified recombinant variants showed a tR equal to 13.583 and 13.747 min for reCG purified by RP-HPLC and HIC, respectively (FIG. 5). These differences in the hydrophobicity of the three molecules could be related with differences in the glycan structures, such as sialic acid content, as they are one of the main structures conferring charges to the proteins.

8.2.2—Spectrofluorometric Analysis

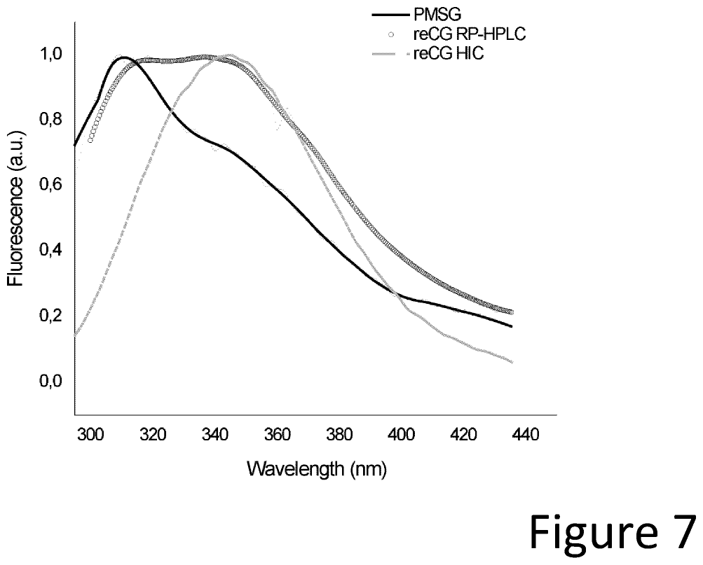

Because the fluorescence profile is extremely sensitive to perturbations in the local structural environment, it provides simple and powerful evidence supporting a high degree of structural similarity between different batches of a given protein. In addition, it can provide useful insights into product comparability and biosimilarity (Houde et al., 2015). Structural conformation assessed through emission profile of the different preparations revealed differences, not only in their maximum peaks but also in their spectra profile. These results should indicate differences in the conformation of the different preparations (FIG. 7).

8.2.3—HPAEC-PAD

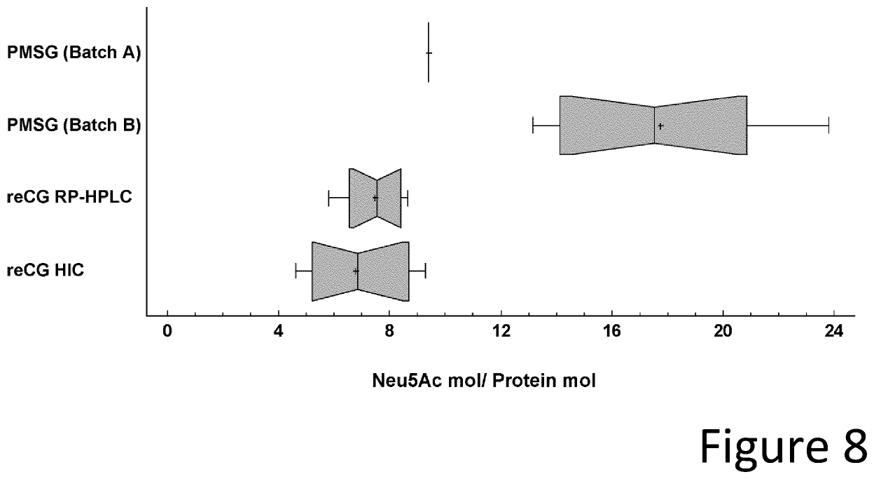

Through HPAEC-PAO (High performance anion exchange chromatography with pulsed amperometric detection) using a OIONEX system, the sialic acid content (Neu5Ac) was evaluated. PMSG (A, Foli-G), PMSG (B, Novormon), reCG RP-HPLC and reCG-HIC exhibited a Neu5Ac content of 9.4 (n=1); 18±4 (n=12); 7±1 (n=11) y 7±2 (n=11) Neu5Acmol/protein mol, respectively. These results correlate with those obtained through RP-HPLC and IEF assays. A non-parametric statistic test (Mood median) allowed determining significant differences between sialic acid content of the different preparations (p: 0.00048). As can be seen in FIG. 8, the notched boxplots showed significant differences between recombinant hormones and PMSG (B, Novormon) at the 95% confidence level.

Despite PMSG preparation displayed higher content of sialic acid than recombinant variants, both PMSG and recombinant forms exhibited nearly the same ratio of sialic acid:galactose, as the proportion of sialic acid and galactose residues of the three hormones were similar

(PMSG (B, Novormon), reCG RP-HPLC and reCG-HIC exhibited a Gal content of 18±0.9 (n=2); 4.7±0.1 (n=2) y 6±0.6 (n=2) Gal mol/protein mol, respectively). Hence, this is one of the attribute that probably favors the lower removal of reCG from circulation, decreasing kidney glomerular filtration and thus, enabling reCG exerts its biological action in the target animal species. Besides, the amount of mannose residues between the hormones were similar, though lesser than expected, as if heterodimer possess three N-glycosylation sites, the amount of Man (mol per mol of protein) should be nearly nine mol/mol. Thus, this lesser contain should be attributed to experimental error. The Fucose contain was similar between the PMSG and recombinant molecules.

8.2.4—Weak Anion Exchange Chromatography (WAX) for Charged Labeled N-Glycans Analysis

To evaluate the sialylation pattern of purified PMSG commercial preparation (B) in comparison with recombinant variants, the N-glycans from each hormone were isolated and labeled with 2-AB. The 2-AB labeled glycans were then applied to a WAX-HPLC column and separated according to their charge, and. to some extent, according with the N-glycan structure. Glycans were identified as neutral- (asialo-), mono-, bi-, tri-, and tetra-sialylated structures using proper standards.

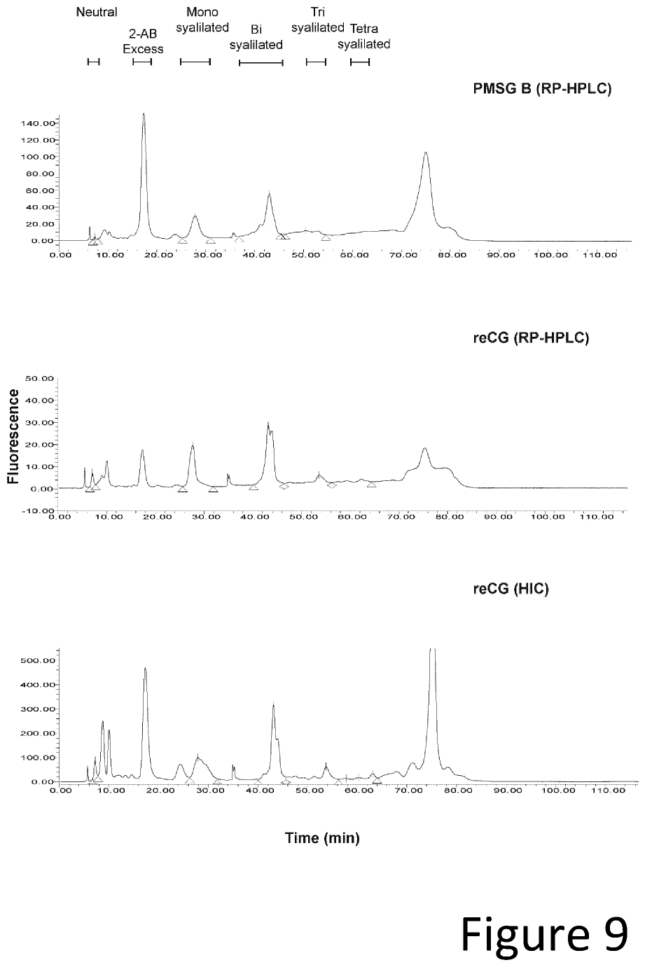

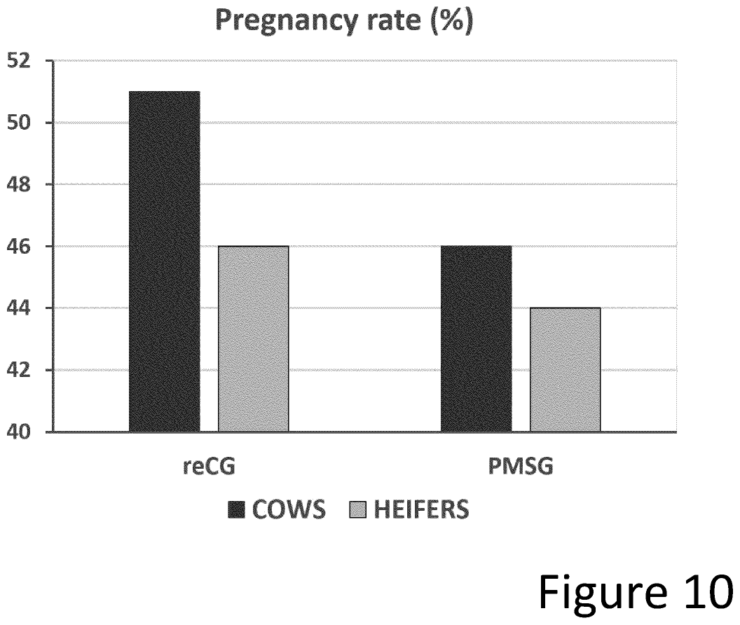

As can be seen in FIG. 9, the N-glycan profiles were similar for the different preparations. However, recombinant preparations exhibited higher content of neutral glycans and tetra-sialylated glycans in comparison with PMSG. Besides, the PMSG preparations showed higher amount of bi-sialylated glycans completely substituted with sialic acid residues (peak corresponding to the last position, from left to right of each group), unlike recombinant variants that exhibited incomplete bi-sialylated structures (and probably more complex).

The reCG RP-HPLC molecule exhibited 3.09% of neutral structures, 30.19% of mono-siaylated, 54.22% bi-sialylated, 8.65% tri-sialylated and 3.86% tetra-sialylated structures. The reCG HIC contained 3.65% of neutral structures, 26.76% mono-sialylated, 52.91% bi-sialylated, 13.53% tri-sialylated and 3.86% tetra-sialylated structures. Finally, the PMSG preparation exhibited the following percentages: 0.72% neutral structures, 29.02% mono-sialylated, 57.99% bi-sialylated and 12.20% tri-sialylated structures (Table 4).

TABLE 4
Relative amount of charged N-glycans of purified
reCG molecules and PMSG.

| | PMSG B | reCG | reCG |
|---|---|---|---|
| N-glycans (%) | (RP-HPLC) | (RP-HPLC) | (HIC) |
| Neutral | 0.72 | 3.09 | 3.65 |
| Mono-sialylated | 29.09 | 30.19 | 26.76 |
| Bi-sialylated | 57.99 | 54.22 | 52.91 |
| Tri-sialylated | 12.20 | 8.65 | 13.53 |
| Tetra-sialylated | — | 3.86 | 3.15 |

### Example 9—Efficacy Assays in Target Animals

9.1—reCG-Induced Superovulation in Heifers

The use of a single-dose eCG in superovulation and embryo production protocols has been exhibited similar efficacy to that obtained by applying multiple doses of FSH. The aim of the present study was to evaluate the efficacy of reCG in inducing superovulation. Eighteen heifers between 350 and 370 kg were synchronized using the following protocol: Day −10, the heifers were injected with 150 μg of PGF2a (Ciclar, ZOOVET); Day 0, they received a intravaginal device with 1200 mg P4 (IVD, Diprogest 1200, ZOOVET) plus a 2 mg EB injection (estradiol benzoate, ZOOVET); Day 4, the animals were randomly divided into 4 groups and injected with: Group 1 (n=4): 1000 IU of reCG; Group 2 (n=4): 1500 IU of reCG; Group 3 (n=5): 2000 IU of reCG and Group 4 (n=4): 2500 IU of PMSG; Day 6, 150 μg of PGF2a (Ciclar, ZOOVET) were injected; Day 7, the IVD was withdrawn and the dose of PGF2a was repeated; Day 8, 0.02 mg of buserelin acetate (ZOOVET) were applied. Ultrasounds (US) were performed on days −10 and 0, to determine the stage of the estrous cycle at the start of the protocol. In addition, on day 8, US was performed to evaluate the number of follicles greater and smaller than 8 mm (FOL<8; FOL>8, respectively) and the number of corpora lutea (CL), and US Doppler (Mindray Z6Vet) was carried out to evaluate the irrigation of follicles>8 mm. All the data obtained were analyzed by ANOVA followed by Duncan post hoc test. The variables irrigation, FOL>8 and CL were then correlated using the Pearson correlation test (SPSS Statistics 23, IBM). Results are summarized in Table 5. Statistical differences were observed between groups 1 and 3 regarding the amount of FOL>8 and CL (P<0.05). Follicle irrigation was positively correlated with FOL>8 and CL (P<0.05). As it is deduced from the results, reCG showed a dose-response effect with respect to the production of preovulatory follicles and CL concomitantly with the increase of the dose. A 2000 IU dose of reCG proved to be as effective as PMSG in inducing superovulation in heifers. Finally, it was demonstrated that the higher follicular irrigation was correlated with a greater number of preovulatory follicles and a greater number of CL.

TABLE 5
Mean value and standard error of the mean of FOL <8, FOL >8, CL and irrigation of
heifers superovulated with reCG and PMSG (n = 18).

| | GROUP | | | | |
|---|---|---|---|---|---|
| | GROUP 1 | GROUP 2 | GROUP 3 | GROUP 4 | |
| | reCG | reCG | reCG | PMSG | |
| | IUeCG | | | | |
| | 1000 | 1500 | 2000 | 2500 | |
| | N | | | | P |
| | 4 | 4 | 5 | 5 | VALUE |
| FOL <8 | 6.40 ± 2.66 | 4.20 ± 1.02 | 5.75 ± 1.38 | 1.5 ± 0.5 | 0.256 |
| FOL >8 | 2.60b ± 0.75 | 7.20ab ± 2.48 | 11.80a ± 1.49 | 7.00ab ± 0.71 | 0.012 |
| CL | 2.2oc ± 0.74 | 4.8obc ± 1.39 | 9_5oa ± 2.26 | 7.30ab ± 0.63 | 0.011 |
| IRRIGATION | 32.60b ± 12.36 | 88.83b ± 19.14 | 176.oa ± 29.2 | 78.5b ± 6.2 | 0.001 |

Values with different superscript letters in a column are significantly different (P < 0.05)

9.2—Pregnancy Tests Assays Using reCG

9.2.1—TFAI: Traditional Protocol

Traditional protocol of 8 consecutive days of P4-releasing intravaginal device.

Day 0: Diagnostic US of the ovarian status of the animals and conformation of the groups under test. Application of an intravaginal device with progesterone 750 mg (Prociclar, Zoovet) plus a 2 mg ES injection (Zoovet).

Day 8: Device withdrawal followed by injections of 150 ug D+Cloprostenol (Ciclar, Zoovet), 1 mg Estradiol Cypionate (Zoovet) and 140 IU reCG or 400 IU PMSG, according to the group. Heat detector paint was applied at the base of the tail of all cows to determine the occurrence or not of mounting (heat) prior to the fixed-time artificial insemination (FTAI).

Day 10 (48 h after removal of the device): Fixed-Time Artificial Insemination

Day 40 (Day 30 from the Al): Diagnostic ultrasonography of pregnancy or cyclicity was performed.

Ultrasonography time schedule: A transrectal ultrasound was performed at time 0 to determine the ovarian status of the females under test. Then, an ultrasound was done on day 40 (30 post-insemination), to obtain the pregnancy diagnosis.

Semen and inseminator: To avoid possible fertility differences outside the protocol that could influence the result, all females were inseminated with the same semen from the same bull and the same batch of straws, and the insemination was carried out by the same professional.

RESULTS: A summary of the results obtained during the process and of the ultrasound on day 30 after fixed-time artificial insemination (FTAI) is reported.

9.2.2—J-Synch FTAI Protocol

Day 0: Diagnostic US of the ovarian status of the animals and conformation of the groups under test. Application of an intravaginal device with progesterone 600 mg (Diprogest, Zoovet) plus a 2 mg Estradiol Benzoate (EB) injection (Zoovet).

Day 6: Device withdrawal followed by injections of 150 ug D+Cloprostenol (Ciclar, Zoovet) and 105 IU reCG or 300 IU PMSG, according to the group. Heat detector paint was applied at the base of the tail of all cows to determine the occurrence or not of mounting (heat) prior to the fixed-time artificial insemination (FTAI).

Day 9 (72 h after removal of the device): All animals were injected with 0.010 mg Buserelin acetate (Zoovet) followed by Fixed-Time Artificial Insemination.

Day 43 (Day 34 from the TFAI): Ultrasonography for pregnancy diagnosis

Ultrasonography time schedule: A transrectal ultrasound was performed at time 0 to determine the ovarian status of the females under test. Then, an ultrasound was done on day 43 (34 post-insemination), to obtain the pregnancy diagnosis.

Semen and inseminator: To avoid possible fertility differences outside the protocol that could influence the result, all females were inseminated with the same semen from the same bull and the same batch of straws, and the insemination was carried out by the same professional.

TABLE 6
Pregnancy results in heifers that were supplemented with PMSG and reCG upon removal of the device in a TFAI protocol

| | | Cattle | | Initial | IU Dose | reCG | | | | PMSG | | | |
|---|---|---|---|---|---|---|---|---|---|---|---|---|---|
| Place | Protocol | Breed | N | CL | reCG/PMSG | n | Pregnant | P % | Final CL | n | Pregnant | P % | Final CL |
| Tacurales | Convent | J × | 103 | 80% | 105/300 | 54 | 25 | 46% | 100% | 49 | 21 | 43% | 100% |
| (Santa Fe) | | HA | | | | | | | | | | | |
| Canada Rosquin | Convent | HA | 67 | N/D | 140/400 | 30 | 17 | 57% | N/D | 37 | 20 | 54% | N/D |
| (Santa Fe) | | | | | | | | | | | | | |
| Palacios | J-SYNCH | HA | 22 | 78% | 140/400 | 13 | 9 | 69% | 100% | 9 | 8 | 89% | 100% |
| (Santa Fe) | | | | | | | | | | | | | |
| Macia | Convent | AA | 90 | N/D | 105/300 | 45 | 22 | 49% | S/D | 45 | 24 | 53% | S/D |
| (Entre Rios) | | | | | | | | | | | | | |
| Mojones Sur | J-SYNCH | AA | 225 | 100% | 105/300 | 149 | 62 | 42% | S/D | 50 | 13 | 26% | S/D |
| (Entre Rios) 1 | | | | | | | | | | | | | |
| Mojones Sur | J-SYNCH | AA | 198 | 100% | 105/300 | 148 | 65 | 44% | S/D | 50 | 19 | 38% | S/D |
| (Entre Rios) 2 | | | | | | | | | | | | | |
| | TOTAL= | | 705 | | | 439 | 200 | .46′)£ | | 240 | 105 | 44′6. | |

| OBSERVED | Pregnant | Empty | | | |
|---|---|---|---|---|---|
| reCG | 200 | 239 | 439 | | |
| PMSG | 105 | 135 | 240 | | |
| | 305 | 374 | 679 | | |
| EXPECTED | | | | CHI SQUARE | |
| | | | | STATISTIC | |
| reCG | 197 | 242 | | CRITICAL VALUE | 3.84 |
| PMSG | 108 | 132 | | P VALUE | 0.6507 |

TABLE 7
Pregnancy results in calving and dry cows that were supplemented with PMSG
(400 IU) and reCG (140 IU) upon removal of the device in an TFAI protocol

| | | Cattle | | | Initial | reCG | | | | PMSG | | | |
|---|---|---|---|---|---|---|---|---|---|---|---|---|---|
| Place | Category | Breed | Protocol | N | CL | n | Pregnant | P % | Final CL | N | Pregnant | P % | Final CL |
| Las Albahacas | dd | AA | Convent | 100 | 20% | 50 | 16 | 32% | N/D | 50 | 17 | 34% | N/D |
| (Cordoba) | | | | | | | | | | | | | |
| Goya | cc | Br | Convent | 41 | 0% | 20 | 9 | 45% | 85% | 21 | 9 | 43% | 62% |
| (Corrientes) | | | | | | | | | | | | | |
| Goya | cc | Br | Convent | 43 | 0% | 33 | 9 | 27% | 48% | 10 | 3 | 30% | 50% |
| (Corrientes) | | | | | | | | | | | | | |
| Tala Huasi | cc | AA | J-SYNCH | 49 | 35% | 25 | 13 | 52% | 100% | 24 | 12 | 50% | 100% |
| (Cordoba) | | | | | | | | | | | | | |
| Humberto | DC | Br | Convent | 48 | 35% | 24 | 12 | 50% | 67% | 24 | 7 | 29% | 67% |
| (Santa Fe) | | | | | | | | | | | | | |
| Mal bran (Sgo del | DC | BrC | Convent | 237 | N/D | 122 | 72 | 59% | N/D | 115 | 61 | 53% | N/D |
| Estero) I | | | | | | | | | | | | | |
| Mal bran (Sgo del | DC | BrN | Convent | 136 | N/D | 68 | 32 | 47% | N/D | 68 | 33 | 49% | N/D |
| Estero) II | | | | | | | | | | | | | |
| Mojones Sur | DC | AA | Convent | 173 | N/D | 120 | 71 | 59% | N/D | 20 | 10 | 50% | N/D |
| (Entre Rios) | | | | | | | | | | | | | |
| | TOTAL= | | | 827 | | 462 | 234 | 51% | 75% | 332 | 152 | 46% | 70% |

| OBSERVED | Pregnant | Empty | | | |
|---|---|---|---|---|---|
| reCG | 234 | 228 | 462 | | |
| PMSG | 152 | 180 | 332 | | |
| | 386 | 408 | 794 | CHI SQUARE | 1.82 |
| | | | | STATISTIC | |
| | | | | CRITICAL VALUE | 3.84 |
| EXPECTED | 225 | 237 | | p VALUE | 0.17 |
| reCG | 161 | 171 | | | |

1. A method for obtaining a mammalian cell line that expresses a recombinant equine chorionic gonadotropin (reCG) hormone comprising the following steps:
a. providing a coding sequences for reCG a and 13 subunits, substantially similar to SEQ ID No 1 and SEQ ID No 2, respectively, optimized for their expression in mammalian cells;
b. introducing said coding sequences in lentiviral expression vectors;
c. producing lentiviruses containing reCG coding sequence;
d. transducing mammalian cells with the above-mentioned lentiviruses;
e. selecting the most suitable mammalian cell clone to produce reCG.

2. The method of claim 1, wherein said cell line comprises a reCG production of at least 100 IU/ml in serum-free medium.

3. The method of claim 1, wherein said step b, said lentiviral vectors comprise the pLV lentiviral vector that contains EF-1a promoter.

4. The method of claim 1, wherein said step c comprises transient transfections of HEK293 cells with pREV, pVSVG, pMDL, pLV-reCG a and pLV-reCG 13 plasmids using cationic lipids as vehicles.

5. The method of claim 1, wherein said step d comprises transducing CHO-K1 cells.

6. The method of claim 5, wherein it comprises two successive transductions.

7. A mammalian CHO cell line obtainable by the method of claim 1 wherein it comprises a nucleic acid that encodes a recombinant equine chorionic gonadotropin (reCG) hormone where the coding sequences for alpha and beta subunits of said reCG comprise sequences substantially similar to SEQ ID No 1 and SEQ ID No 2.

8. The cell line of claim 7, wherein it is CHO-K1 cell line.

9. The cell line of claim 7, wherein it produces at least 100 IU/ml reCG.

10. A method for producing a recombinant equine chorionic gonadotropin (reCG) hormone comprising the following steps:
a. cultivating of said mammalian cell line of claim 7 in bioreactor in serum-free medium for large-scale production of reCG
b. harvesting the supernatant, and
c. purification.

11. The method of claim 10, wherein it comprises a productivity of at least 100 IU/ml reCG in serum-free medium.

12. The method of claim 10, wherein said step “a” comprises cultivation in serum-free medium containing 50% commercial Excel302 medium and 50% phosphate-buffered saline.

13. The method of claim 10, wherein said purification step c., comprises a dye pseudo-affinity chromatography.

14. The method of claim 13, wherein said dye pseudo-affinity chromatography uses CaptoBlue-Sepharose matrix.

15. The method of claim 10, wherein also comprises a HPLC purification step using a C4 column.

16. (canceled)

17. The method of claim 10, wherein said reCG comprises a specific activity (as in vivo potency units in relation to the protein mass determined by ELISA) of at least 6000 IU/mg.

18-21. (canceled)

22. A pharmaceutical formulation comprising a therapeutically effective amount of the reCG of claim 20.

23. (canceled)

24. The pharmaceutical formulation of claim 22, wherein it is liquid, and wherein it further comprises a sugar, a preservative, an antioxidant, mannitol, and an anti-aggregation agent and wherein the pharmaceutical composition is kept chilled at 5° C., without the need to be frozen.

25. (canceled)

26. The pharmaceutical formulation of claim 22, wherein the pharmaceutical formulation is liquid and wherein it comprises trisodium citrate dihydrate, citric acid monohydrate, arginine, sucrose, mannitol, L-methionine, poloxamer 188, m-cresol and water.

27. A method for inducing ovulation in animals comprising the administration of at least 140 IU/animal of the reCG obtainable by claim 10.

28. (canceled)
